(19) United States
(12) Patent Application Publication
(10) Pub. No.: US 2026/0105987 A1
(43) Pub. Date: Apr. 16, 2026

(54) USING PROTEIN LARGE LANGUAGE MODELS TO IMPROVE PROTEIN ACTIVITY

(71) Applicant: Massachusetts Institute of Technology, Cambridge, MA (US)

(72) Inventors: Omar Abudayyeh, Cambridge, MA (US); Jonathan Gootenberg, Cambridge, MA (US); Kaiyi Jiang, Cambridge, MA (US); Matteo Di Bernardo, Cambridge, MA (US)

(73) Assignee: Massachusetts Institute of Technology, Cambridge, MA (US)

(21) Appl. No.: 19/143,784

(22) PCT Filed: Oct. 11, 2024
(86) PCT No.: PCT/US24/50915

§ 371 Date: Jun. 26, 2025

Related U.S. Application Data
(60) Provisional application No. 63/590,139, filed on Oct. 13, 2023.
(60) Provisional application No. 63/672,398, filed on Jul. 17, 2024.

(51) Int. Cl. G16B 30/00; G16B 40/20

(52) U.S. Cl. CPC G16B 30/00; G16B 40/20

(57) ABSTRACT

An initial protein sequence is identified and varied to generate a plurality of variants. An activity of the plurality of protein sequences is measured quantitatively. Each of the plurality of protein sequences is provided as an input to a large language model (LLM) to generate a corresponding plurality of embeddings in a latent space. A subset of the plurality of embeddings is used to train a top layer model. The plurality of embeddings are provided as inputs to the top layer model to generate outputs representing predictions of the activity of the plurality of protein sequences. A subset of the plurality of protein sequences is selected based on the top layer model outputs. The method repeats, with the selected subset of the plurality of protein sequences playing the role of the initial protein sequence in the initial iteration, until some termination criterion is satisfied.

### BACKGROUND

Measuring the activity of proteins, including enzymes such as those involved in genome editing, has traditionally been rooted in tedious and time-consuming experimental benchwork. The indel activity of enzymes involved in genome editing, such as Cas9 or Cas12, is typically gauged by expression in cells, inducing the desired activity (e.g., making targeted cuts in the DNA), harvesting the cells, and subjecting them to assays. To find protein sequences with improved activity, traditional approaches involve making rational changes to the protein sequence based on known functional domains or using methods such as directed evolution, which involves inducing random mutations in the protein's gene, selecting for improved function, and iteratively repeating this process.

These existing techniques have a variety of drawbacks. For example, they because such techniques are time-consuming, performed manually, and require multiple steps of cell culturing, transfection, incubation, and subsequent analyses, only a limited number of protein variants can be tested in parallel, making wide-scale optimization challenging. Such techniques also have empirical limitations. For example, methods such as directed evolution rely on random mutations, which means that a vast sequence space remains unexplored. Although rational design can target known domains, it is constrained by current knowledge, potentially overlooking unforeseen beneficial mutations. Furthermore, such techniques are resource intensive and can produce inconsistent results.

In light of these limitations of existing techniques for exploring protein activity, there is a need for more efficient, consistent, and wide-reaching methods for selecting mutants on which to perform experimental testing.

### SUMMARY

Improved methods and systems for predicting and improving protein activity are disclosed. An initial protein sequence is identified and varied to generate a plurality of variants. An activity of the plurality of protein sequences is measured quantitatively. Each of the plurality of protein sequences is provided as an input to a large language model (LLM) to generate a corresponding plurality of embeddings in a latent space. A subset of the plurality of embeddings is used to train a top layer model. The plurality of embeddings are provided as inputs to the top layer model to generate outputs representing predictions of the activity of the plurality of protein sequences. A subset of the plurality of protein sequences is selected based on the top layer model outputs (e.g., the top-ranking protein sequences). The method repeats, with the selected subset of the plurality of protein sequences playing the role of the initial protein sequence in the initial iteration. The process repeats until some termination criterion is satisfied.

Other features and advantages of various aspects and embodiments of the present invention will become apparent from the following description and from the claims.

### BRIEF DESCRIPTION OF THE DRAWINGS

FIG. 1 is a flowchart of an example method for predicting and improving protein activity according to one embodiment of the present invention.

### DETAILED DESCRIPTION

Improved methods and systems for predicting and improving protein activity are disclosed. An initial protein sequence is identified and varied to generate a plurality of variants. An activity of the plurality of protein sequences is measured quantitatively. Each of the plurality of protein sequences is provided as an input to a large language model (LLM) to generate a corresponding plurality of embeddings in a latent space. A subset of the plurality of embeddings is used to train a top layer model. The plurality of embeddings are provided as inputs to the top layer model to generate outputs representing predictions of the activity of the plurality of protein sequences. A subset of the plurality of protein sequences is selected based on the top layer model outputs (e.g., the top-ranking protein sequences). The method repeats, with the selected subset of the plurality of protein sequences playing the role of the initial protein sequence in the initial iteration. The process repeats until some termination criterion is satisfied.

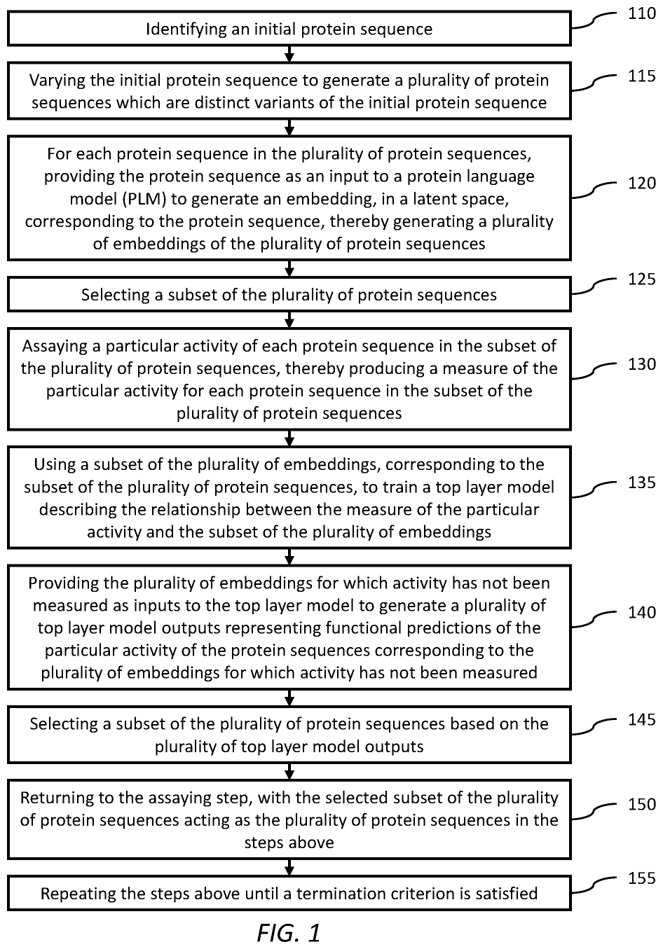

FIG. 1 is a flowchart of an example method for predicting and improving protein activity according to one embodiment of the present invention. An initial protein sequence (e.g., a wild type protein sequence) may be identified (e.g., selected or generated) (FIG. 1, step 110). Such a protein sequence may be of any of a variety of types. As one example, the protein sequence may be a Cas12f protein sequence.

The initial protein sequence may be varied (e.g., in silico) to generate a plurality of protein sequences which are distinct variants (e.g., mutants) of the initial protein sequence (FIG. 1, step 115). The plurality of protein sequences may be of the same type as the initial protein sequence. For example, if the initial protein sequence is a Cas12 protein sequence, then the plurality of protein sequences may include variants of the Cas12 protein sequence. Varying the initial protein sequence may include, for example, performing any one or more of the following operations on the initial protein sequence: inserting one or more amino acids into the initial protein sequence, deleting one or more amino acids from the initial protein sequence, substituting one or more amino acids for one or more amino acids in the initial protein sequence, or rearranging one or more amino acids in the initial protein sequence.

Varying the initial protein sequence in silico may include introducing a plurality of distinct point mutations (i.e., substitutions of a single amino acid) into the initial protein sequence to generate the plurality of protein sequences (e.g., a plurality of variants of the wild type protein sequence). Such point mutations may occur at different points in the initial protein sequence. As another example, varying the initial protein sequence may include varying the initial protein sequence with a predetermined upper bound of variations relative to a wild type of the initial protein sequence. As yet another example, varying the initial protein sequence may include randomly choosing an index within the protein at which to vary the initial protein sequence.

For each protein sequence in the plurality of protein sequences, the protein sequence is provided as an input to a large language model (LLM) to generate an embedding, in a latent space, corresponding to the protein sequence (FIG. 1, step 120). This results in a plurality of such embeddings corresponding to the plurality of protein sequences. The LLM may, for example, be an LLM that has been trained on protein sequences, which is referred to herein as a protein language model (PLM). An example of such a PLM is the Evolutionary Scale Modeling (ESM) protein language model available from Facebook Research.

A subset of the plurality of embeddings may be selected (e.g., using some selection method) (FIG. 1, step 125). For example, the initial subset of the plurality of embeddings may be selected randomly.

One or a plurality of particular activities of the subset of the plurality of protein sequences may be assayed (FIG. 1, step 130). When assaying multiple activities, such activities may be assayed and optimized simultaneously. Some non-exhaustive examples of these activities are indel activity (e.g., indel activity relative to a mammalian genome), activity between the protein sequence and a chemical compound or small molecule, activity between the protein sequence and another protein, activity between the protein sequence and a peptide, activity between the protein sequence and a sugar, activity between the protein sequence and a nucleic acid, activity between a protein and lipid or lipid nanoparticles, and activity between a protein and salt or metal ions.

The assaying may include assaying the particular activity of each protein sequence in the subset of the plurality of protein sequences, thereby producing a measure of the particular activity for each protein sequence in the subset of the plurality of protein sequences. Each such measure may be a quantitative measure, such as a single numerical value for each protein sequence in the subset of the plurality of protein sequences. For example, a value of a quantitative function may be measured for each protein sequence in the subset of the plurality of protein sequences. Such quantitative function values are examples of the measures of the particular activity.

A subset of the plurality of embeddings, corresponding to the subset of the plurality of protein sequences, may be used to train a top layer model that describes the relationship between the measure of the particular activity and the subset of the plurality of embeddings (FIG. 1, step 135). The number of embeddings in the subset may be significantly smaller than the number of embeddings in the plurality of embeddings. For example, the subset of the plurality of embeddings may consist of in the range of 8-32 embeddings, while the plurality of embeddings may consist of hundreds or thousands of embeddings. The top layer model may, for example, be a regression model, a random forest model, a deep learning model, a convolutional neural network, or a multilayer perceptron.

While the LLM may be domain-agnostic, the top layer model may be trained on domain-specific task inputs, as described in more detail below.

The plurality of embeddings for which activity has not been measured may be provided as inputs to the top layer model to generate a plurality of top layer model outputs representing functional predictions of the activity of protein sequences corresponding to the plurality of embeddings for which activity has not been measured (FIG. 1, step 140). Such functional predictions may, for example, include predictions of fluorescence intensity or thermal stability. Before providing the plurality of embeddings for which activity has not been measured as inputs to the top layer model, the dimension of those embeddings may be reduced to produce reduced-dimension embeddings, and the reduced-dimension embeddings may be provided as the inputs to the top layer model.

The plurality of top layer model outputs may, for example, include a plurality of vectors representing quantitative biological functions.

Since, as described above, the total number of embeddings in the plurality of embeddings may be significantly larger than the number of embeddings in the subset of the plurality of embeddings, the number of embeddings that are provided as inputs to the top layer model, and the corresponding number of outputs of the top layer model, may be significantly larger than the number of embeddings that were used to train the top layer model.

A subset of the plurality of protein sequences may be selected based on the plurality of top layer model outputs (or a subset of the top layer model outputs, such as only those top layer model outputs that were generated based on the plurality of embeddings for which activity has not been measured) (FIG. 1 step 145). For example, the top layer model outputs may be ranked (e.g., in descending order), and some number (e.g., anywhere between 8 and 32) of the top-ranked outputs (and their corresponding protein sequences) may be selected. Note that while the initial subset of the plurality of protein sequences may have been selected randomly or in some other way because the top layer model had not yet been trained, once the top layer model has been trained its outputs may be used to select subsets of the plurality of protein sequences in subsequent rounds of the method.

The method may return to the assaying step, with the selected subset of the plurality of protein sequences acting as the plurality of protein sequences in the steps above (FIG. 1, step 150). For example, in the next iteration of the varying step, each protein sequence in the selected subset of the plurality of protein sequences may be varied in the manner described above, and that iteration of the method may proceed using the resulting variations.

The method may continue to iterate as described above until some termination (e.g., convergence) criterion is satisfied (FIG. 1, step 155). The method may, for example, be terminated manually when the operator observes that the method is not producing variants with significantly better-performing variants.

Embodiments of the present invention may be used in connection with any of a variety of proteins, of which the following are examples:

### 1. Receptor-Ligand:

a. β-adrenergic receptors and agonists/antagonists: Used in heart failure, asthma (e.g., albuterol).
b. Muscarinic acetylcholine receptors and agonists/antagonists: Used in Alzheimer's disease, overactive bladder (e.g., donepezil, oxybutynin).
c. Dopamine receptors and agonists/antagonists: Used in Parkinson's disease, schizophrenia (e.g., levodopa, haloperidol).
d. EGFR (Epidermal Growth Factor Receptor): Therapeutic antibodies (e.g., cetuximab) and small molecule inhibitors (e.g., erlotinib) for cancer.
e. BCR-ABL
f. Estrogen Receptor (ER): Tamoxifen and raloxifene for breast cancer and osteoporosis prevention.
g. Progesterone Receptor (PR): Mifepristone is used for abortive purposes.
h. Glucocorticoid Receptor (GR): Corticosteroids like prednisone for inflammation and autoimmune diseases.
i. GABAA receptor: Benzodiazepines (e.g., diazepam) and barbiturates for anxiety and sleep disorders.
j. NMDA receptor: Memantine for Alzheimer's disease and ketamine for depression.
k. α4β7 integrin: Vedolizumab is an antibody used for inflammatory bowel disease.
l. Programmed Death Ligand-1 (PD-L1) and its Receptor PD-1:
m. HER2/neu (a receptor tyrosine kinase) and its Ligands:
n. Angiotensin II and its Receptor (AT1R):
o. Endothelin-1 and its Receptor (ETAR, ETBR):

### 2. Cytokine Receptors:

IL1R, IL2R, IL6R, IL12R, IL17R, IL23R, TNF-alpha receptor, GM-CSFR, IL4R, IL5R, IFNR

### 3. Transcription Factors:

STAT family, NF-kb family, P53 family, WNT family, MYC family, HIF-1 family.
   4. Kinase and phosphatase:
BCR-ABL, ZAP70, SYK, ALK, BRAF, MEK, C-KIT, PI3K, JAK, CDK, PP2A, SHP1/2, PTEN, CDC25, Calcineurin.

### 5. Polymerase:

T7, Pfu, Taq, Phi29 DNA polymerase, KOD, Q5, Klenow Fragment

### 6. Integrase and Recombinase:

HIV-1 integrase, PhiC31, Bxb1, TN7, FLP, Cre.

### 7. Transposon:

TN3, TN5, TN10, Class II P elements, Mariner/TC1, Sleeping beauty, PiggyBac, LINE1.

The method described above has been implemented in a model called EVOLVE-Pro (Evolutionary-scale Protein Optimization). EVOLVE-Pro is an ensemble model for few-shot learning to evolve proteins, combining evolutionary-scale protein language models with a top-layer discrimination model to learn a protein's functional landscape and guide the directed evolution process in silico. While the method described herein may be referred to as “EVOLVE-Pro” in certain contexts, it should be understood that the use of this name is for convenience and clarity only. The term “EVOLVE-Pro” should not be interpreted to limit the scope of the specification or claims in this patent application to any particular implementation or description of the model referred to as “EVOLVE-Pro” in other publications or documents. The claims and specification of this patent application define the scope of the invention, regardless of how the method may be named or described elsewhere. The methods and systems disclosed herein may be implemented in various ways and may evolve over time, and the protection sought is not limited to any specific implementation known as “EVOLVE-Pro” at the time of filing or at any other time.

Embodiments of the method described herein evolve protein variants with enhanced activity using minimal experimental testing and a few-shot learning approach, enabling accurate prediction of sequence-to-function relationships for general properties. This performance may be achieved through an ensemble approach that combines large-scale protein language models with a top-layer discrimination model to comprehend a protein's functional landscape and guide the directed evolution process computationally. By implementing this method in a few-shot, active learning framework, protein sequences with significantly improved activity may be efficiently identified in a generalizable manner with minimal experimental effort. The modular architecture of this method allows for scalability with increasingly complex protein language models. Furthermore, the method requires only protein sequences as input for evolution, without the need for structural information, expert knowledge, or prior data. The multi-modal nature of this approach enables simultaneous engineering of multiple protein features of any type or data class, presenting extensive possibilities for applications in biology and medicine.

To establish EVOLVE-Pro, we designed an ensemble model that involves: 1) a foundational protein language model to encode protein sequences into an information-rich latent space, and 2) a top-layer discrimination model to learn protein functional grammar in this evolutionary landscape and rank protein sequences according to a designed policy framework, and 3) an active learning framework using top layer discrimination model to nominate the next set of protein variants for experimental evaluation. This cycle is performed iteratively to evolve defined protein activities until they reach desired levels.

We optimized EVOLVE-Pro across five parameters: 1) the strategy employed for the first round mutant selection, 2) the top layer discrimination model that learns the fitness landscape, 3) the active learning strategy for selecting mutants for the next round, 4) the evolution policy, and 5) the embedding vector transformation (Table S1). To perform a grid search across this space, we curated a panel of twelve unique deep mutagenesis scanning (DMS) datasets for in silico validation (16-26) (Table S2). These twelve proteins represent diverse functions, including viral spike proteins, RNA-guided nucleases, lactases, and kinases, ensuring that the resulting model will be as generalizable as possible for learning diverse protein activity landscapes in PLM latent space.

TABLE S1
Summary of parameters grid search

| Fitness | First- | Active | PLM | Top | | Mutant |
|---|---|---|---|---|---|---|
| measurement | Round | learning | embedding | layer | Round | Nomination |
| data type | strategy | strategy | types | model | Number | numbers |
| Raw input | Random | Random | Residue | Ridge | 5 | 10 |
| | | | Averaged | regression | | |
| Min-Max scaled | Max | Top N | PCA of | Lasso | 10 | 20 |
| input | distance K- | | Residue | regression | | |
| | medoids | | Averaged | | | |
| | clusters | | (Top 10 | | | |
| | | | PCs) | | | |
| | | Top N/2 + | | Elastic | | 30 |
| | | Bottom | | Net | | |
| | | N/2 | | | | |
| | | Max | | Multilayer | | 40 |
| | | Euclidean | | perceptron | | |
| | | distance | | | | |
| | | | | Linear | | 50 |
| | | | | Regression | | |
| | | | | Neural | | 100 |
| | | | | Network | | |
| | | | | Random | | 200 |
| | | | | Forest | | |
| | | | | Regressor | | |
| | | | | XGboost | | 500 |

TABLE S2
Description for 12 DMS datasets

| | | | | | | | Efficient | |
|---|---|---|---|---|---|---|---|---|
| | | | | | | | evolution | |
| | | | | | | | rate | |
| | | | Fitness | | Population | Population | (hie et al | Background |
| Protein | Reference | Organism | setting | Cutoff | successes | size | 2023) | (%) |
| ADRB2 | Jones et | Human | Signal | >2.8 | 914 | 7800 | 22 | 12 |
| | al., | | transduction + | | | | | |
| | 2019 | | pathway | | | | | |
| | | | reporter | | | | | |
| β- | Stiffler | Bacteria | Antibiotic | >0.01 | 393 | 4978 | 40 | 7.9 |
| lactamase | et al, | | resistance | | | | | |
| | 2015 | | (ampicillin, | | | | | |
| | | | 2500 ug/mL) | | | | | |
| Env | Haddox | Virus | Viral | >0.1 | 748 | 12863 | 23 | 5.8 |
| | et al., | | replication | | | | | |
| | 2016 | | fitness | | | | | |
| HA H1 | Doud and | Virus | Viral | >0.1 | 645 | 10716 | 16 | 6 |
| | Bloom, | | replication | | | | | |
| | 2016 | | fitness | | | | | |
| HA H3 | Lee et al., | Virus | Viral | >0.1 | 714 | 10754 | 31 | 6.6 |
| | 2018 | | replication | | | | | |
| | | | fitness | | | | | |
| infA | Kelsic | Bacteria | Competitive | >0.98 | 305 | 1368 | 50 | 22 |
| | et al., | | growth | | | | | |
| | 2016 | | | | | | | |
| MAPK1 | Brenan | Human | Competitive | >2.5 | 77 | 6810 | 7.7 | 1.1 |
| | et al., | | growth | | | | | |
| | 2016 | | (SCH772984) | | | | | |
| P53 | Giacomelli | Human | Competitive | >1 | 905 | 7448 | 12 | 12 |
| | et al, | | growth | | | | | |
| | 2018 | | (etoposide) | | | | | |
| PafA | Markin | Bacteria | Kcat/KM | P < 0.01, | 35 | 1040 | 20 | 3.4 |
| | et al., | | | faster | | | | |
| | 2021 | | | than WT | | | | |
| AsCas | Hino et al., | Bacteria | Genomic | >1 | 1436 | 7941 | 28 | 18 |
| 12f | 2023 | | DNA | | | | | |
| | | | Cleavage | | | | | |
| Zika | Sourisse | Virus | Viral | >1 | 351 | 9576 | 12 | 3.7 |
| Envelope | au et al., | | replication | | | | | |
| protein | 2019 | | fitness | | | | | |
| COV2 | Greaney | Virus | Yeast | >0.05 | 232 | 1959 | 0 | 12 |
| Spike | et al., | | display | | | | | |
| Protein | 2021 | | binding | | | | | |

We first focused on the ESM-2 protein language model because of its large training data and available model size of >200M proteins and 15B parameters, respectively. Using the ESM-2 15B parameter model, our grid search found the optimal strategy was: 1) selecting a random set of first-round variants, 2) employing a random forest regressor discriminatory model to predict protein function, 3) using residue pooled average embeddings, and 4) using a top-N selection strategy in each round of evolution. This policy nominated a high frequency of gain-of-function protein variants in only 5 rounds. Since we focused on percent of activity passing a threshold as our evaluation metric in the grid search, we next checked for increasing function during in silico evolution. We found that both the median activity and the activity of the nominated top mutant increased monotonically from round to round across all DMS datasets, further validating the model's performance in this low-N active learning setting.

In general, 16 mutants per round of evolution for 10 rounds identified top mutants with fitness in the 50th percentile for eleven of the twelve DMS datasets. To understand how the number of variants per round affected performance, we tested between 10 and 100 variants per round, finding that larger rounds increased prediction accuracy. This performance trade off indicates that EVOLVE-Pro can be used for both extremely low-N evolution (<20 mutants per round) for rapid and cheap experimental characterization and medium-N (˜100 mutants per round) for quicker and more efficient evolution with fewer rounds.

After optimizing the top layer model and learning strategies, we optimized the PLM, comparing ESM-2 15B to a panel of foundational models. Using the optimal parameters from the grid search, we benchmarked performance against smaller versions of ESM-2 and ESM-1(27), UniRep(15, 28), ProtT5(29), ProteinBERT(4), Ankh(3), one-hot encoding, and integer encoded protein representations. ESM-2 15B parameter model outperformed all the other models for identifying the highest fitness proteins for all datasets except two, confirming its final selection for the EVOLVE-Pro latent space model. Importantly, large parameter PLMs showed a significant boost in prediction accuracy compared to non-language model-based architectures, indicative of the powerful feature extraction present in transformer-based models.

We next benchmarked EVOLVE-Pro's performance relative to other PLM-based engineering approaches. As many methods require pre-training a discriminatory model on thousands of variants, we tested versions of EVOLVE-Pro augmented with various amounts of pre-training. Reinforcement learning drastically reduced the overall number of mutants required: EVOLVE-Pro with only 5 rounds of evolution (16 mutants per round) was equivalent in performance to EVOLVE-Pro pre-trained with 160 mutants, while 10 rounds of evolution (16 mutants per round) was equivalent to pre-training with 500 mutants. Moreover, EVOLVE-Pro significantly outperformed zero-shot prediction methods (30). This comparison confirms that the few-shot nature of EVOLVE-Pro allows for efficient directed evolution with minimal effort and low-N testing per round.

Lastly, we analyzed the per-round evolution improvement for EVOLVE-Pro compared to one-hot and integer encoding and zero-shot prediction, finding that by round 5 variants with significantly enhanced fitness could universally be found (at 16 mutations per round). Moreover, in many cases, the one-hot and integer encoding frameworks saturated much earlier in the evolution process and never reached the fitness levels achieved by EVOLVE-Pro. Interestingly, for some proteins we observe a non-linear increase in protein fitness after round 3, suggesting greater gains in mapping the protein fitness landscape as EVOLVE-Pro evolution proceeds.

Antibody Optimization with EVOLVE-Pro

The EVOLVE-Pro model may be applied to optimize binding interactions of antibodies, such as the binding of the REGN10987 antibody to the extracellular epitope of the SARS-CoV-2 spike protein. This application demonstrates the model's capability to improve proteins that have been challenging to optimize through previous in silico methods. The EVOLVE-Pro method may involve mutagenizing specific regions of a protein, such as the heavy chain variable region of an antibody. In each round of optimization, the model may nominate multiple mutant variants for experimental testing. These variants may be compared to the wild-type protein using appropriate assays, such as enzyme-linked immunosorbent assays (ELISA) for antibody-antigen interactions.

One feature of EVOLVE-Pro is its ability to achieve significant improvements in protein function within a small number of optimization rounds. The success rate of the model typically increases with each round, demonstrating the model's capacity to learn and improve its predictions over time. This improvement in performance illustrates that the top layer model learns a functional grammar that is distinct from the fitness grammar captured initially by the underlying foundational model. This distinction allows EVOLVE-Pro to identify beneficial mutations that may not be predicted by traditional evolutionary models.

To understand EVOLVE-Pro's mutational trajectory, the model's attention to particular residues may be analyzed. This analysis often reveals that the model repeatedly explores multiple residues, with successive rounds of training focusing on specific regions of the protein. This behavior emphasizes EVOLVE-Pro's ability to identify and concentrate on functionally important areas of the protein sequence.

The function of variants predicted by the model often does not correlate with the fitness predicted by the protein language model (PLM) alone. This lack of correlation can be observed by comparing the observed activity of each mutant to its PLM-predicted fitness score. To further explore this phenomenon, the base layer PLM fitness score and the top layer random forest predicted functional improvement can be projected in the latent space for all possible single mutation variants. This analysis typically reveals little overlap between the two distributions, often showing a negative correlation between predicted fitness and predicted function.

These findings collectively demonstrate that protein language models do not learn protein function in isolation, highlighting the importance of the few-shot learning approach employed by EVOLVE-Pro. By combining the strengths of large-scale protein language models with a specialized top layer model, EVOLVE-Pro can efficiently identify protein variants with improved function, even when those improvements are not predicted by fitness scores alone.

### Evolution of a Miniature RNA-Guided CRISPR Nuclease

Programmable RNA-guided nucleases have diverse applications in basic biology, therapeutics, and diagnostics. However, commonly used nucleases, such as the Cas9 from Streptococcus pyogenes (SpCas9) are too large to effectively be packaged in common viral vectors such as adeno-associated viral (AAV), and more compact high-efficiency nucleases, such as the Cas9 from Staphylococcus aureus (SaCas9) still preclude the use of larger regulatory elements or protein fusions. Miniature Cas12f nucleases have compact sizes (<700 residues) but suffer from reduced efficiencies, requiring significant engineering for genome editing applications. Previous Cas12f engineering efforts relied on DMS or rationally designed mutations to increase the in vitro cleavage activity, requiring extensive screening to find the optimal variant. To accelerate miniature nuclease engineering, we tested whether EVOLVE-Pro could rapidly develop highly active Cas12f variants.

To understand the mechanisms of beneficial mutations nominated by EVOLVE-Pro, structural prediction tools such as AlphaFold may be used to analyze the protein structure. This approach can provide insights into how the mutations nominated by the protein language model (PLM) may contribute to enhancing the protein's activity. The structural analysis may reveal potential effects of mutations on various protein domains, such as changes in binding affinity, stabilization of secondary structures, or alterations in protein conformation.

The EVOLVE-Pro model's attention to particular residues in the protein may be analyzed by calculating the cumulative frequency of individual residues explored by the model. This analysis often reveals that multiple residues are repeatedly nominated by the model. To understand the relationship between the base layer PLM's fitness prediction and the actual measured protein activity, the predicted marginal masked score (pMMS) can be calculated for each nominated mutant. Importantly, EVOLVE-Pro often nominates higher activity mutants in later rounds that are contrary to high fitness mutants recommended by the PLM base layer alone. This demonstrates a valuable feature of EVOLVE-Pro: the ability to distinguish between predicted fitness and actual function. By projecting both the base layer PLM's fitness score and the top layer random forest regressor's activity score in the ESM2 latent space, EVOLVE-Pro's global mutational trajectory can be better understood. Typically, a weak correlation is observed between fitness and activity scores, further highlighting the benefits of a top-layer discrimination model to properly distinguish between high fitness and high activity.

Engineering Improved Prime Editors with EVOLVE-Pro

Many molecular tools, such as next-generation genome editing proteins, function as multiple enzymes acting in concert. Prime editing, which uses an RNA-templated reverse transcriptase to programmably install diverse genome edits, is one such tool that combines a CRISPR-Cas nuclease with an engineered reverse transcriptase. The EVOLVE-Pro model can be applied to improve upon rationally designed mutations in such complex molecular tools, potentially offering advantages over other directed evolution approaches. The EVOLVE-Pro method can be particularly useful for addressing challenges in genome editing, such as the difficulty of installing longer edits. For example, the model can be applied to optimize editing outcomes with longer insertions, which have utility for programmable gene insertion methods. To apply EVOLVE-Pro to such systems, an evolution policy may be set up using appropriate experimental approaches. For instance, overlapping guide RNAs may be used in combination to install specific sequences at target genomic loci. The editing efficiency may then be quantified using methods such as amplicon sequencing and next-generation sequencing. The top layer EVOLVE-Pro model may be trained on this data to predict insertion efficiency, allowing for iterative improvement of the editing system.

The EVOLVE-Pro model demonstrates the ability to progressively learn the activity landscape of complex molecular tools over successive rounds of optimization. This iterative learning process allows the model to yield improved variants after the initial random selection round, with substantial improvements observed in later rounds of evolution. To validate the generalizability of the model's predictions, the top-performing variants may be evaluated across multiple genomic loci in different cell lines. Such evaluations typically reveal statistically significant improvements in editing efficiency across diverse genomic contexts, highlighting the model's capacity to enhance overall protein activity rather than optimizing for a single specific target. This approach underscores EVOLVE-Pro's potential to deliver broadly applicable improvements in protein function, particularly for complex molecular tools involving multiple enzymatic components.

The EVOLVE-Pro method may be used in conjunction with structural prediction tools to analyze potential beneficial mutations. This approach can provide insights into how mutations nominated by the model might contribute to enhancing protein activity, including unexpected effects in different protein domains. One feature of EVOLVE-Pro is its ability to analyze residue site preferences during evolution. The model often demonstrates significant attention to specific residues, suggesting it learns which positions are most beneficial for improving activity. This learning process occurs over multiple rounds of evolution. Analysis of predicted fitness scores from the bottom layer protein language model (PLM) typically reveals a divergence between fitness and activity predictions for the evolved protein. This divergence allows EVOLVE-Pro to successfully use its top discrimination layer to navigate towards higher activity variants in later evolution rounds, even when these variants may not be predicted to have high fitness by the base PLM alone. This capability highlights a benefit of EVOLVE-Pro: its ability to distinguish between predicted fitness and actual function, potentially identifying beneficial mutations that traditional evolutionary models might overlook.

To understand EVOLVE-Pro's global mutational trajectory, the activity landscape learned by the random forest regressor and the base layer protein language model's fitness landscape may be projected onto the principal components of the embedding space. This analysis typically reveals a significant divergence between these two landscapes, often showing little to no convergence between their distributions. The lack of convergence between the activity and fitness landscapes is frequently characterized by a weak or even negative correlation. This finding underscores a beneficial aspect of EVOLVE-Pro's functionality: its ability to distinguish between predicted fitness and actual functional activity of protein variants.

Bxb1 Integrase Evolution with EVOLVE-Pro

Large serine recombinases (LSRs) are enzymes that facilitate precise DNA rearrangements, making them crucial tools for genome editing. Their ability to recognize specific DNA sequences and catalyze targeted recombination events allows for efficient and accurate modifications of genetic material, which is essential for advanced gene therapy, synthetic biology, and genetic research. However, LSRs often have limitations in their activity levels within cells, which can restrict the overall efficiency of genetic modifications. The EVOLVE-Pro model may be applied to improve the activity of LSRs, potentially enhancing their performance for various genome editing applications. The EVOLVE-Pro method may be used to address specific challenges in LSR functionality, such as improving their activity in cellular environments where their efficiency may be limited. By applying EVOLVE-Pro to LSRs, researchers can aim to overcome these limitations and achieve higher levels of recombination efficiency, potentially expanding the utility of these enzymes in genome editing technologies.

To apply EVOLVE-Pro to large serine recombinases, an integration assay may be designed to measure the enzyme's activity. This assay typically involves the insertion of DNA sequences containing specific recognition sites into target plasmids or genomic loci. The integration efficiency can be quantified using next-generation sequencing techniques, providing a measurable output for the EVOLVE-Pro model to optimize. The evolution process using EVOLVE-Pro generally involves multiple rounds of optimization. In each round, the model nominates variants for experimental testing. Over successive rounds, a progressive increase in enzyme activity is typically observed. This iterative process allows the model to learn and refine its predictions, potentially leading to significant improvements in enzyme performance. To validate the results of the evolution campaign, top-performing variants may be tested under various conditions, such as different expression levels or in alternative cell lines. This validation process helps ensure that the improvements in enzyme activity are robust and generalizable across different experimental contexts.

To evaluate the broader applicability of variants identified by EVOLVE-Pro, the model's output may be tested in various genomic contexts. For example, improved enzyme variants can be assessed for their ability to enhance the programmable insertion of DNA cargo into multiple genomic loci. Such testing typically reveals consistent improvements in insertion efficiency across diverse genomic targets, demonstrating the generalizability of the activity enhancements achieved through EVOLVE-Pro optimization. This approach underscores EVOLVE-Pro's capacity to produce enzyme variants with broadly applicable improvements, rather than optimizations specific to a single genomic context or experimental condition.

Structural prediction tools may be used in conjunction with EVOLVE-Pro to analyze the potential effects of mutations identified by the model. This approach can provide insights into how the nominated mutations might contribute to enhancing protein activity, such as improving DNA binding affinity for enzymes that interact with nucleic acids. EVOLVE-Pro demonstrates the ability to identify mutations in functionally important regions of proteins, such as DNA-binding domains. This capability highlights the model's potential to optimize key aspects of protein function without relying solely on random mutagenesis. An important feature of EVOLVE-Pro is its analysis of residue exploration during the evolution process. The model often revisits certain positions multiple times throughout the optimization rounds, indicating its ability to recognize and focus on residues that are particularly important for improving protein function. Overall, EVOLVE-Pro's approach to identifying functionally important regions in proteins is comparable to structure-guided engineering methods. This similarity underscores the model's sophisticated understanding of protein structure-function relationships, enabling it to make informed predictions about beneficial mutations.

EVOLVE-Pro's analysis includes examining the relationship between predicted fitness (as determined by protein language models) and observed functional improvements. This analysis often reveals varying degrees of correlation between these metrics for different protein families. In some cases, protein families may show a correlation between predicted fitness and observed activity. However, even when such correlations exist, they are often weak, highlighting the limitations of relying solely on fitness predictions for protein engineering. The EVOLVE-Pro model demonstrates its value by efficiently identifying high-performing variants, even in cases where there is only a weak correlation between predicted fitness and observed function. This capability allows EVOLVE-Pro to navigate the protein sequence space effectively, minimizing false positives and accelerating the discovery of improved variants. A key strength of EVOLVE-Pro is its ability to learn protein function at a global scale. The model's learned mutation landscape often diverges significantly from fitness predictions made by protein language models alone. This divergence underscores EVOLVE-Pro's capacity to capture complex relationships between sequence and function that may not be apparent from stability or fitness predictions. Consequently, EVOLVE-Pro offers a more comprehensive approach to protein evolution, potentially leading to more rapid and efficient improvements in protein function compared to methods relying solely on stability or fitness predictions.

### Evolving T7 RNA Polymerase for Highly Pure and Efficient RNA Production

EVOLVE-Pro demonstrates versatility in evolving enzymes for multiple objectives simultaneously. This capability is particularly useful for optimizing enzymes involved in complex processes with multiple performance parameters. When applying EVOLVE-Pro to multi-objective optimization, an objective function may be designed to incorporate multiple parameters of interest. These parameters may be weighted according to their relative importance for the desired application. For example, in optimizing an RNA polymerase, parameters such as RNA yield, translation efficiency, and RNA purity may be included in the objective function.

EVOLVE-Pro's optimization process may involve multiple rounds of evolution. Initial rounds may show modest improvements, while later rounds often yield more significant enhancements across multiple parameters simultaneously. This demonstrates the model's ability to navigate complex fitness landscapes and identify synergistic mutations.

EVOLVE-Pro may generate multi-mutant variants by combining mutations identified in earlier rounds. Unlike traditional rational mutagenesis approaches that rely on assumptions about synergistic effects, EVOLVE-Pro learns the activity landscape to nominate multi-mutants in an unbiased fashion. This approach can reveal unexpected epistatic effects between residues at different spatial positions in a protein.

EVOLVE-Pro can identify unique mechanisms for improving enzyme function that may not be apparent through rational design approaches. The model can explore mutations in various regions of the protein, including those not typically considered in structure-guided engineering. During the evolution process, EVOLVE-Pro demonstrates an ability to identify functionally important residues. The model often revisits these key residues in subsequent rounds, exploring additional mutations in the surrounding regions. This focused exploration allows EVOLVE-Pro to efficiently optimize protein function.

EVOLVE-Pro's analysis often reveals a divergence between observed functional improvements and fitness predictions from protein language models. The model successfully navigates this divergence by selecting variants with higher activity even when they may not have high predicted fitness. This highlights EVOLVE-Pro's ability to capture complex relationships between sequence and function that may not be apparent from stability or fitness predictions alone.

EVOLVE-Pro demonstrates the ability to learn protein function at a global scale. The mutational landscape learned by EVOLVE-Pro often diverges significantly from fitness predictions made by protein language models alone. This underscores EVOLVE-Pro's capacity to identify beneficial mutations that might be overlooked by methods relying solely on fitness or stability predictions.

EVOLVE-Pro can be applied to optimize enzymes for the production of complex RNA structures, such as circular RNA. This demonstrates the model's versatility in addressing challenges in advanced RNA synthesis techniques. When applied to RNA polymerase optimization, EVOLVE-Pro can simultaneously enhance multiple aspects of enzyme performance, such as reducing unwanted byproduct formation (e.g., double-stranded RNA) while improving transcription fidelity and overall RNA yield.

EVOLVE-Pro-optimized enzymes may significantly improve the efficiency of circular RNA production. This can be observed through increased translation efficiency of the resulting circular RNA and reduced formation of immunogenic byproducts. The model can be used to evolve enzymes that maintain high fidelity during extended transcription periods, which is crucial for processes like circular RNA production. This demonstrates EVOLVE-Pro's ability to optimize enzymes for specific, demanding applications.

Enzymes optimized by EVOLVE-Pro can be evaluated using various analytical techniques, such as gel electrophoresis, fluorescence imaging, and specialized assays (e.g., dsRNA ELISA). These methods can confirm improvements in RNA quality, yield, and reduced byproduct formation.

EVOLVE-Pro-optimized enzymes may lead to significant improvements in the overall efficiency of RNA production processes. This can include higher yields of the desired RNA product, improved purity, and reduced formation of unwanted byproducts. By optimizing enzymes for complex RNA synthesis applications, EVOLVE-Pro demonstrates its capacity to generate variants with properties that may not be easily achievable through traditional protein engineering approaches.

EVOLVE-Pro can be applied to optimize enzymes for the production of mRNA with specific modifications, such as those used in in vivo imaging or therapeutic applications. This demonstrates the model's versatility in addressing challenges in advanced mRNA synthesis techniques. Enzymes optimized by EVOLVE-Pro may significantly enhance the quality and performance of produced mRNA. This can be observed through increased translation efficiency and prolonged expression of the encoded proteins in vivo.

The model can be used to evolve enzymes that maintain high fidelity during the production of modified mRNAs, which is valuable for applications such as in vivo imaging or therapeutic mRNA production. This demonstrates EVOLVE-Pro's ability to optimize enzymes for specific, demanding applications that mimic clinical production processes.

Enzymes optimized by EVOLVE-Pro can be evaluated in contexts that closely resemble their intended applications. For example, mRNA produced by these optimized enzymes can be tested in vivo using appropriate delivery methods, such as lipid nanoparticles, to assess their performance in relevant biological systems.

EVOLVE-Pro-optimized enzymes may lead to significant improvements in the performance of mRNA in vivo. This can include higher levels of protein expression and prolonged duration of expression, which are important factors for applications such as in vivo imaging or mRNA therapeutics.

By optimizing enzymes for complex mRNA synthesis applications, EVOLVE-Pro demonstrates its capacity to generate variants with properties that may significantly outperform wild-type enzymes in terms of mRNA quality and in vivo performance.

### EVOLVE-Pro for Biological and Drug Development Applications

EVOLVE-Pro is an ensemble model designed for few-shot learning to evolve proteins. Through iterative rounds of improvement, EVOLVE-Pro can yield variants with significant enhancements in desired properties, including binding affinity, catalytic efficiency, and reduction of immunogenic byproducts. The model leverages evolutionary scale protein language models to learn general rules about protein function and employs a discriminatory interpretation model layer to reason protein designs with improved activity. This approach enables rapid evolution of diverse proteins.

EVOLVE-Pro's architecture, which combines a rich latent space generated by protein language models with powerful feature selection in the top layer module, allows for a low-N learning approach. This means the model requires minimal human intervention and experimentation to achieve results. The effectiveness of EVOLVE-Pro has been demonstrated across multiple protein classes and datasets, showcasing its superiority in low-N evolution settings.

The model's design incorporates a comprehensive evaluation of embedding-based protein language models and utilizes a large parameter grid search to optimize various aspects of the approach. This includes examining different discriminatory and active learning selection strategies, as well as various normalization techniques for embeddings and fitness measurements. Importantly, the research found that protein language models are essential for high-quality representations of protein sequences, as dimensionality reduction of the embedding space through techniques like PCA did not improve performance.

EVOLVE-Pro's modular design allows for the integration of future improvements in autoregressive protein language models, potentially scaling up the model's capabilities towards de novo generation of highly active mutants. The model has demonstrated its effectiveness in engineering proteins that are challenging to evolve using existing machine learning-directed evolution approaches, particularly those with low correlation between activity and fitness or those requiring simultaneous optimization of multiple properties.

A key advantage of EVOLVE-Pro is its compatibility with a wide range of protein activity assays, including those that are not amenable to pooled screening methods typically used in directed evolution strategies. This broadens the applicability of the model to a diverse set of protein engineering challenges.

While evolutionary scale protein language models (PLMs) have demonstrated effectiveness in various protein analysis tasks, they face challenges in predicting protein sequences with superior function. This limitation stems from the PLMs' training objective, which focuses on learning evolutionary representations rather than maximizing protein function. As a result, the evolutionary landscape learned by PLMs often diverges from a protein's functional landscape. EVOLVE-Pro addresses this limitation by incorporating a discriminatory interpretation model layer on top of the PLM. This additional layer allows the model to bridge the gap between evolutionary representations and functional improvements, enabling more effective protein engineering across a wide range of protein types.

The model's approach is particularly valuable for engineering enzymes, which present unique challenges due to their complex biophysical and biochemical properties. Unlike antibodies, where PLM-based evolution may have some success due to the primarily biophysical nature of antigen binding, enzymes require simultaneous optimization of multiple properties and often involve poorly understood catalytic mechanisms.

EVOLVE-Pro's design accounts for the limitations of current PLMs, including the observed saturation in performance improvements as model size increases. By combining PLM-generated embeddings with a specialized top layer model, EVOLVE-Pro can effectively navigate the complex relationship between protein sequence and function, potentially overcoming the limitations of PLMs alone in predicting and optimizing protein activities.

While generative approaches to protein design have shown promise, they often face limitations in producing functional proteins with high activity. EVOLVE-Pro addresses these challenges by combining the strengths of generative models with a specialized optimization framework. Unlike purely generative methods, EVOLVE-Pro's approach allows for the efficient optimization of protein sequences towards specific functional objectives. This is particularly valuable in cases where generative models alone may produce sequences with lower activity compared to naturally occurring proteins or existing engineered variants.

EVOLVE-Pro's design incorporates lessons learned from both deep learning and non-deep learning approaches to protein engineering. By leveraging a protein language model for initial sequence representation and combining it with a specialized top layer model, EVOLVE-Pro can potentially outperform both generative protein language models and traditional sequence diversification methods in certain scenarios.

EVOLVE-Pro operates in a “high p, low N” paradigm, leveraging high-dimensional protein embeddings while requiring only a small number of new data points in each experimental evolution round. This approach allows the model to efficiently navigate complex protein sequence spaces with minimal experimental input. The model employs a random forest regressor as its top layer, which provides several advantages in this context. The random forest algorithm inherently imposes regularization and accounts for covariation between independent variables, making it well-suited for handling the high-dimensional, low-sample-size data characteristic of protein engineering problems.

EVOLVE-Pro's active learning strategy utilizes a top-n selection of variants, which has proven to be highly effective. This approach not only guides the evolution process but also allows users to observe round-by-round improvements in real-time, providing valuable insights into the optimization trajectory. The model's design is flexible and can potentially incorporate future advancements in machine learning and active learning strategies. For instance, Bayesian-driven frameworks could be integrated to quantify uncertainty in protein predictions, potentially leading to more informed variant selection. However, such approaches would need to be carefully balanced against the current strategy's advantage of providing real-time feedback on improvements.

EVOLVE-Pro demonstrates robust performance in out-of-distribution protein engineering, extending beyond benchmark datasets. The model has shown consistent success in improving protein function through novel residue mutations, with later rounds of evolution yielding highly active variants. The model's effectiveness has been demonstrated across diverse protein classes, including antibodies, genome-editing enzymes, and polymerases. In validation experiments, EVOLVE-Pro-generated variants have achieved state-of-the-art performance compared to both wild-type and previously engineered variants.

EVOLVE-Pro's capabilities extend to pre-clinical experimental settings, including animal models, highlighting its potential for rapid translation of computational predictions to practical applications. This demonstrates the model's ability to generate variants with improved performance in complex biological systems. Analysis of top mutations identified by EVOLVE-Pro has provided insights into mechanisms of activity improvement. These findings can guide future protein engineering efforts and suggest novel approaches for enhancing enzyme function. For example, the model has identified potential improvements in domains not typically targeted in traditional engineering approaches. Overall, EVOLVE-Pro showcases the ability to produce engineered proteins with state-of-the-art performance while also providing valuable insights into novel mechanistic directions for protein improvement.

EVOLVE-Pro represents a significant advancement in protein engineering models. Unlike generative protein design models that often have low success rates and require high-quality initial inputs, EVOLVE-Pro offers several key advantages, such as high success rates in protein optimization; no requirement for special knowledge about the target protein; capability for multi-objective function optimization; and multi-modal functionality, allowing the use of any quantifiable property as input. These features enable EVOLVE-Pro to efficiently optimize proteins across multiple properties simultaneously. The model's effectiveness has been demonstrated in its ability to navigate vast sequence spaces, selecting highly active single mutants from over 16,000 possible sequences and multi-mutants from more than 780 billion possible sequences.

While current protein function datasets are limited in their coverage of the complete protein design space, EVOLVE-Pro serves as a powerful tool for protein engineering within existing constraints. The model's approach allows for efficient exploration and optimization of protein sequences without requiring exhaustive datasets. EVOLVE-Pro is positioned as a general protein engineering tool accessible to users in biology and drug development. Its design allows for protein engineering with minimal effort and cost, making it a valuable resource for a wide range of applications in these fields. As the field of protein engineering advances, EVOLVE-Pro's flexible architecture positions it to incorporate future developments in high-throughput data collection and analysis. This adaptability suggests potential for even more comprehensive protein design capabilities in the future.

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions.

Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

Embodiments of the present invention include features which are only possible and/or feasible to implement with the use of one or more computers, computer processors, and/or other elements of a computer system. Such features are either impossible or impractical to implement mentally and/or manually. For example, embodiments of the present invention may generate embeddings in a latent space using a large language model. Such a function is inherently rooted in computer technology and cannot be performed mentally or manually. As another example, embodiments of the present invention may measure an activity of a plurality of sequences in vitro. Such a function cannot be performed mentally and is not an abstract idea, or is a practical application of an abstract idea.

Any claims herein which affirmatively require a computer, a processor, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

Any step or act disclosed herein as being performed, or capable of being performed, by a computer or other machine, may be performed automatically by a computer or other machine, whether or not explicitly disclosed as such herein. A step or act that is performed automatically is performed solely by a computer or other machine, without human intervention. A step or act that is performed automatically may, for example, operate solely on inputs received from a computer or other machine, and not from a human. A step or act that is performed automatically may, for example, be initiated by a signal received from a computer or other machine, and not from a human. A step or act that is performed automatically may, for example, provide output to a computer or other machine, and not to a human.

The terms “A or B,” “at least one of A or/and B,” “at least one of A and B,” “at least one of A or B,” or “one or more of A or/and B” used in the various embodiments of the present disclosure include any and all combinations of words enumerated with it. For example, “A or B,” “at least one of A and B” or “at least one of A or B” may mean: (1) including at least one A, (2) including at least one B, (3) including either A or B, or (4) including both at least one A and at least one B.

Although terms such as “optimize” and “optimal” are used herein, in practice, embodiments of the present invention may include methods which produce outputs that are not optimal, or which are not known to be optimal, but which nevertheless are useful. For example, embodiments of the present invention may produce an output which approximates an optimal solution, within some degree of error. As a result, terms herein such as “optimize” and “optimal” should be understood to refer not only to processes which produce optimal outputs, but also processes which produce outputs that approximate an optimal solution, within some degree of error.

What is claimed is:

1. A method comprising:
identifying an initial protein sequence;
varying the initial protein sequence to generate a plurality of protein sequences which are distinct variants of the initial protein sequence;
for each protein sequence in the plurality of protein sequences, providing the protein sequence as an input to a large language model (LLM) to generate an embedding, in a latent space, corresponding to the protein sequence, thereby generating a plurality of embeddings of the plurality of protein sequences;
selecting a subset of the plurality of protein sequences;
assaying a particular activity of each protein sequence in the subset of the plurality of protein sequences, thereby producing a measure of the particular activity for each protein sequence in the subset of the plurality of protein sequences;
using a subset of the plurality of embeddings, corresponding to the subset of the plurality of protein sequences, to train a top layer model describing the relationship between the measure of the particular activity and the subset of the plurality of embeddings;
providing the plurality of embeddings for which activity has not been measured as inputs to the top layer model to generate a plurality of top layer model outputs representing functional predictions of the particular activity of the protein sequences corresponding to the plurality of embeddings for which activity has not been measured;
selecting a subset of the plurality of protein sequences based on the plurality of top layer model outputs;
returning to the assaying step, with the selected subset of the plurality of protein sequences acting as the plurality of protein sequences in the steps above;
repeating the steps above until a termination criterion is satisfied.

2. The method of claim 1, wherein the particular activity comprises an indel activity of each of the plurality of protein sequences relative to a mammalian genome.

3. The method of claim 1, wherein the particular activity comprises at least one of:
an activity between each of the plurality of protein sequences and a chemical;
an activity between each of the plurality of protein sequencies and another protein;
an activity between each of the plurality of protein sequencies and a peptide;
an activity between each of the plurality of protein sequencies and a sugar; or
an activity between each of the plurality of protein sequencies and a nucleic acid.

4. (canceled)

5. (canceled)

6. (canceled)

7. (canceled)

8. The method of claim 1, wherein varying the initial protein sequence comprises introducing a plurality of distinct point variations into the initial protein sequence to generate the plurality of protein sequences.

9. The method of claim 1, wherein varying the initial protein sequence comprises varying the initial protein sequence with a predetermined upper bound of variations relative to a wild type of the initial protein sequence.

10. The method of claim 1, wherein varying the initial protein sequence comprises randomly choosing an index within the protein at which to vary the initial protein sequence.

11. The method of claim 1, wherein the LLM comprises an LLM that has been trained on protein sequences.

12. The method of claim 1, wherein the LLM is domain-agnostic and wherein the top layer model is trained on domain task-specific inputs.

13. The method of claim 1, wherein the top layer model comprises a regression model.

14. The method of claim 1, wherein the top layer model comprises a random forest model.

15. The method of claim 1, wherein the top layer model comprises a deep learning model.

16. The method of claim 1, wherein the top layer model comprises a convolutional neural network.

17. (canceled)

18. (canceled)

19. The method of claim 1, wherein the functional predictions comprise predictions of fluorescence intensity.

20. The method of claim 1, wherein the functional predictions comprise predictions of thermal stability.

21. The method of claim 1, wherein the plurality of top layer model outputs comprises a plurality of vectors representing quantitative biological functions.

22. The method of claim 1, wherein selecting the subset of the plurality of protein sequences comprises:
ranking the plurality of protein sequences based on the plurality of top layer model outputs; and
selecting the top-ranked plurality of protein sequences as the subset.

23. The method of claim 1, further comprising:
before providing the plurality of embeddings for which activity has not been measured as inputs to the top layer model, reducing the dimension of the plurality of embeddings for which activity has not been measured to produce a plurality of reduced-dimension embeddings, and providing the plurality of reduced-dimension embeddings to the top layer model.

24. The method of claim 1, wherein the initial protein sequence comprises a Cas12 protein sequence, and wherein the plurality of protein sequences comprises a plurality of Cas12 protein sequences.

25. The method of claim 1, wherein measuring the particular activity of the plurality of protein sequences comprises measuring the activity of the plurality of protein sequence in vitro.

26. The method of claim 1, wherein using the subset of the plurality of embeddings to train the top layer model comprises using a selection method to select the subset of the plurality of embeddings.
